(19) United States
(12) Patent Application Publication
(10) Pub. No.: US 2019/0300789 A1
(43) Pub. Date: Oct. 3, 2019

(54) METHOD FOR STORING AND TRANSPORTING CHLOROALKANES

(71) Applicant: BLUE CUBE IP LLC, Clayton, MO (US)

(72) Inventors: Max Tirtowidjojo, Clayton, MO (US); Marc Sell, Clayton, MO (US); John D. Myers, Clayton, MO (US)

(21) Appl. No.: 16/373,226

(22) Filed: Apr. 2, 2019

Related U.S. Application Data
(60) Provisional application No. 62/652,087, filed on Apr. 3, 2018.

(51) Int. Cl. C09K 15/08; C09K 15/20; C09K 15/18

(52) U.S. Cl. CPC C09K 15/08; C07C 19/05; C09K 15/18; C09K 15/20

(57) ABSTRACT

Disclosed herein are inhibitor mixtures that prevent or minimize the degradation of halogenated alkanes. The formulations contain acid acceptor stabilizer and/or a metal inhibitor. Methods of making and using the inhibitor mixture are also described.

### CROSS REFERENCE TO RELATED APPLICATIONS

The instant application claims the benefit of U.S. Provisional Patent Application Ser. No. 62/652,087, filed Apr. 3, 2018, which is incorporated herein by reference in its entirety.

### FIELD OF THE INVENTION

The present disclosure generally relates to compositions that inhibit the degradation of chloroalkanes during shipping and storage, and processes for preparing and using the compositions.

### BACKGROUND OF THE INVENTION

Chlorinated alkanes are useful intermediates for many products including agricultural products, pharmaceuticals, cleaning solvents, gums, silicones, and refrigerants. Accordingly, chlorinated alkanes are made on an industrial scale and are often stored before being shipped to a purchaser. But, chlorinated alkanes are known to degrade over time by interacting with the metal containing walls of the storage container and/or any water, oxygen, acid, metal ions, and/or other compounds that may be present. The by-products made by these degradation processes may be one or more of halogenated, oxygenated by-products, such as phosgene. Other degradation products included HCl. The degradation products decrease the purity of the chloroalkane and result in additional, undesired by-products, when the chloroalkane is used in subsequent reactions.

It would be desirable to develop compositions and methods that prevent or reduce the degradation of chlorinated alkanes while stored and/or during shipping.

### SUMMARY OF THE INVENTION

Disclosed herein are compositions that inhibit the degradation of halogenated alkanes. These inhibitory mixtures comprise an acid acceptor stabilizer and/or a metal inhibitor. Other compounds may also be present in the inhibitory mixture.

Also disclosed are methods of using the disclosed inhibitor mixtures to prevent or inhibit the degradation of a halogenated alkane. Such processes comprising: combining a halogenated alkane with an inhibitor mixture comprising an acid acceptor stabilizer and/or a metal inhibitor, wherein the halogenated alkane is optionally dried before it is treated with the inhibitor mixture.

Methods of making the prepared inhibitory mixture are also disclosed.

Other features and iterations of the invention are described in more detail below.

### DETAILED DESCRIPTION OF THE INVENTION

### The Halogenated Alkane

The compositions and methods disclosed herein are used to prevent or inhibit degradation of halogenated alkanes. In on embodiment, the halogenated alkanes are C₁-C₆ halogenated alkanes. Alternatively, the halogenated alkanes are C₂-C₄ halogenated alkanes. Halogenated propanes are one preferred class of compounds that can be treated with the inhibitory mixture disclosed herein.

The halogenated alkanes may be substituted with one or more halogens, which are selected from the group consisting of F, Cl, Br, and I. In one embodiment, the halogenated alkane is substituted with at least one Cl. More preferably, with at least two Cl groups. Alternatively, the halogenated alkane is substituted with at least three Cl groups. In a preferred embodiment, the halogenated alkane is substituted with at least four Cl groups. In an alternate, preferred embodiment, the halogenated alkane is substituted with at least five Cl groups. Specific halogenated alkanes that may be treated using the compositions and methods disclosed herein include 1,1,1,3-tetrachloropropane (HCC-250FB); 1,1,1,-trichloroethane; 1-chloroethane; 1,1,2-trichloroethane; tetrachloroethanes; methyl chloride; dichloromethane; chloroform; carbon tetrachloride; 1,1,1,2,3-pentachloropropane (HCC-240DB); 1,1,2,2,3-pentachloropropane, 1,1,1,3,3,3-hexachloropropane, or 1,1,1,3,3-pentachloropropane. In some embodiments, combinations of more than one of the aforementioned halogenated alkanes may be present. The halogenated alkane may be substituted with a mixture of two or more halogen atoms. Examples include at least one Cl and at least one F.

### The Inhibitory Mixture—the Acid Acceptor

As disclosed above, the inhibitory mixture comprises an acid acceptor compound and/or a metal inhibitor.

The acid acceptor comprises one or more compounds. The acid acceptor compounds are compounds that interact with acids, e.g., HCl, present in the halogenated alkane. Examples of such compounds include amylene, butylene oxide, propylene oxide, cyclohexene oxide, butoxymethyl oxirane, isopropyl acetate, cyclohexane, trimethylpentene, isopropyl alcohol, pyridine, triethylamine, epichlorohydrin, 4-methylmorpholine, n-methylmorpholine, n-methyl pyrrole, nitromethane and combinations of two or more thereof.

### The Inhibitory Mixture—the Metal Inhibitor

The metal inhibitor comprises one or more compounds. The metal inhibitor compounds interact with metal ions that may be present in the halogenated alkane. Examples of metal inhibitors include 1,4-dioxane, cyclohexane, 1,3-dioxolane, isoamyl alcohol, ethyl acetate, formaldehyde dimethyl hydrazone, or 2-methyl-3-buyn-2-ol, furfuryl alcohol, dimethyl oxalate, glycidol, and mixtures thereof.

The inhibitory mixture may contain additional inhibitors, such as comprises a free radical inhibitor, and oxidant inhibitor, or a combination thereof. In one embodiment, the additional inhibitor is a substituted phenol. In another embodiment, examples of such compounds include thymol, 2-methyl-2-butane, phenol, cresol, resorcinol, or a combination of two or more thereof. In a further embodiment, the inhibitor mixture comprises <1% resorcinol, by weight.

### Methods of Preventing or Inhibiting Degradation

The methods of preventing or inhibiting degradation of the halogenated alkanes comprising adding at least one acid acceptor and/or at least one metal inhibitor to the halogenated alkane. The halogenated alkane may be stirred or otherwise mixed before, during, and/or after the inhibitory mixture is added. If the inhibitory mixture contains two or more compounds, the compounds may be combined and then added to the halogenated alkane or the compounds may be sequentially added to the halogenated alkane.

If desired, the halogenated alkane may be dried before it is treated with the inhibitor mixture. Methods known in the art, such as the use of drying agents, may be used. Examples of drying agents include calcium chloride, Al₂O₃, activated carbon, molecular sieve, ion exchange resins or a combination of two or more thereof. Other methods include azeotropic distillation.

Once the halogenated alkane is in a storage or shipping container, it may be padded with an inert gas, i.e., inert gas is added to the container, in an effort to exclude water and/or oxygen. The gas pad may be added to the container after the inhibitory mixture is added, but if desired, the inhibitory mixture may be added after the gas pad is added to the container. In one embodiment, the pressure of the inert gas pad is greater than atmospheric pressure. If desired, the container may be lined with an inert material, before the halogenated alkane is added. Inert materials are non-iron and non-aluminum containing materials. Examples of inert materials include Teflon and glass.

The halogenated alkane containing the inhibitory mixture may be stored or shipped at a temperature of about 0° C. to about 60° C. More preferably, the temperature of the halogenated alkane containing the inhibitory mixture is about 10° C. to about 60° C. Still more preferably, the temperature of the halogenated alkane containing the inhibitory mixture is about 20° C.-50° C.

The halogenated alkane containing the inhibitory mixture may be stored or shipped at an elevated pressure. The pressure may be about 0-100 psig. Or the pressure may be about 0-50 psig. Alternatively, the pressure may be about 0-25 psig. In one embodiment, the pressure is about 0-10 psig.

In one embodiment, the inhibitor mixture comprises: ≤4.5 wt % 1,4-dioxane ≤1 wt % 1,2-butylene oxide, and ≤0.9 wt % nitromethane. More preferably, the inhibitor mixture comprises ≤4 wt % 1,4-dioxane ≤0.6 wt % 1,2-butylene oxide, and 0.5 wt % nitromethane. Still more preferably, the inhibitor mixture comprises <4 wt % 1,4-dioxane <0.6 wt % 1,2-butylene oxide, and <0.5 wt % nitromethane.

In another embodiment, the inhibitor mixture comprises: ≤1.5 wt % 1,4-dioxane; ≤2.5 wt % 1,3-Dioxolane; ≤1.5 wt % Amylene(1-Pentene); ≤2.5 wt % propylene oxide, and ≤0.6 wt % cyclohexane. More preferably, the inhibitor mixture comprises ≤1.0 wt % 1,4-dioxane; ≤2 wt % 1,3-Dioxolane; ≤1.0 wt % Amylene(1-Pentene); ≤2 wt % propylene oxide, and <0.1 wt % cyclohexane. Yet more preferably, the inhibitor mixture comprises <1.0 wt % 1,4-dioxane; <2 wt % 1,3-Dioxolane; <1.0 wt % Amylene(1-Pentene); <2 wt % propylene oxide, and <0.1 wt % cyclohexane.

In a further embodiment, the inhibitor mixture comprises: ≤0.9 wt % 2-methyl-2-butane; ≤0.9 wt % phenol; ≤0.9 wt % thymol; ≤0.1 wt % cresol. More preferably, the inhibitor mixture comprises ≤0.5 wt % 2-methyl-2-butane; ≤0.5 wt % phenol; ≤0.5 wt % thymol; ≤0.01 wt % cresol. More preferably, the inhibitor mixture comprises <0.5 wt % 2-methyl-2-butane; <0.5 wt % phenol; <0.5 wt % thymol; <0.01 wt % cresol.

### Processes for Preparing the Inhibitory Mixtures

The inhibitory mixtures described herein may be prepared by a variety of methods. In one method, the acid acceptor stabilizer and the metal inhibitor components are mixed together, simultaneously, and optionally mixed. Then the inhibitory mixture is added to the halogenated alkane. Alternatively, the components are added to the halogenated alkanes sequentially. For example, in one embodiment, the acid acceptor stabilizer component(s) is/are added to the halogenated alkane, followed by the metal inhibitor component(s). Alternatively, the metal inhibitor component(s) is/are added to the halogenated alkane, followed by the acid acceptor stabilizer component(s).

### DEFINITIONS

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### Sample Inhibitor Mixtures

The following examples illustrate various embodiments of the invention. In the following examples, the inhibitor mixture is added to a halogenated alkane. The amounts of the compounds in the inhibitor mixtures are based on weight. The weight of the halogenated alkane plus the weight of the components in the inhibitor mixture total 100 weight %.

### Example 1

50 cc of 99.5 wt % 1,1,1,2,3-pentachloropropane (240DB) containing 300 ppm of water is placed in two brown bottle containers. One bottle sample is padded with air and the cap is opened periodically to exchange the gas phase. The sample bottles are placed in a heated cabinet maintained at 50° C. The other container is padded with N₂. After more than 4 weeks, the analysis shows that the sample padded with air is expected to contain more than 10 ppm of HCl than the ones with the ones padded with N₂.

### Example 2

The experiment of example 1 is repeated, except that to the sample padded with air is added less than 10 ppm of Amylene. After 4 weeks it is expected that the HCl content is similar to or less than the one padded with N₂.

### Example 3

The experiment of example 1 is repeated, but with the addition of a carbon steel (CS) coupon to the sample padded with air. The sample analysis after more than 4 weeks is expected to show much higher level of HCl and iron ions than the base line.

### Example 4

The experiment in example 3 is repeated except that less than 10 ppm of amylene and less than 300 ppm of 1,4-dioxane are added to the sample with the CS coupon. After more than 4 weeks, the analysis is expected to show less HCl and iron ion content than those in the sample with CS coupon in example 3.

What is claimed is:

1. A process for inhibiting degradation of a halogenated alkane, the process comprising: Combining a halogenated alkane with an inhibitor mixture comprising an acid acceptor stabilizer and/or a metal inhibitor, wherein the halogenated alkane is optionally dried before it is treated with the inhibitor mixture.

2. A process according to claim 1, wherein the acid acceptor comprises one or more of compounds comprising amylene, butylene oxide, propylene oxide, cyclohexene oxide, butoxymethyl oxirane, isopropyl acetate, cyclohexane, trimethylpentene, isopropyl alcohol, pyridine, triethylamine, epichlorohydrin, 4-methylmorpholine, n-methylmorpholine, n-methyl pyrrole, nitromethane and combinations of two or more thereof.

3. A process according to claim 1, wherein the metal inhibitor comprises one or more of 1,4-dioxane, cyclohexane, 1,3-dioxolane, isoamyl alcohol, ethyl acetate, formaldehyde dimethyl hydrazone, or 2-methyl-3-buyn-2-ol, furfuryl alcohol, dimethyl oxalate, and glycidol.

4. A process according to claim 1, wherein the halogenated alkane is a chlorinated alkane.

5. A process according to claim 1, wherein the halogenated alkane is dried by treating it with calcium chloride, Al₂O₃, activated carbon, molecular sieve, ion exchange resins or a combination of two or more thereof.

6. A process according to claim 4, wherein the chlorinated alkane is a chlorinated C₁-C₄ alkane.

7. A process according to claim 6, where in the chlorinated C₁-C₄ alkane is a chlorinated propane.

8. A process according to claim 4, wherein the chlorinated alkane comprises at least one of 1,1,1,3-tetrachloropropane; 1,1,1,-trichloroethane; 1-chloroethane; 1,1,2-trichloroethane; tetrachloroethanes; methyl chloride; dichloromethane, chloroform; carbon tetrachloride; 1,1,1,2,3-pentachloropropane; 1,1,2,2,3-pentachloropropane; 1,1,1,3,3,3-hexachloropropane; or 1,1,1,3,3-pentachloropropane.

9. A process according to claim 1, wherein the halogenated alkane is in a container and is padded with an inert gas.

10. A process according to claim 10, wherein the pressure of the inert gas pad is greater than atmospheric pressure.

11. A process according to claim 9, wherein the container is lined with an inert material.

12. A process according to claim 11, wherein the inert material is a non-iron and non-aluminum containing material.

13. A process according to claim 1, wherein the inhibitor mixture further comprises a free radical inhibitor, an oxidant inhibitor, or a combination thereof.

14. A process according to claim 1, wherein the inhibitor mixture further comprises thymol, 2-methyl-2-butane, phenol, cresol, resorcinol, or a combination of two or more thereof.

15. A process according to claim 1, wherein the temperature of the halogenated alkane is about 0 to about 60° C.

16. A process according to claim 1, wherein the pressure of the halogenated alkane is about 0-100 psig.

17. A process according to claim 1, where the inhibitor mixture comprises: <4% 1,4-dioxane <0.6% 1,2-butylene oxide, and <0.5% nitromethane.

18. A process according to claim 1, where the inhibitor mixture comprises: <1.0 wt % 1,4-dioxane; <2 wt % 1,3-dioxolane; <1.0 wt % amylene(1-pentene); <2 wt % propylene oxide, and <0.1 wt % cyclohexane.

19. A process according to claim 1, where the inhibitor mixture comprises: <0.5 wt % 2-methyl-2-butane; <0.5 wt % phenol; <0.5 wt % thymol; and <0.01 wt % cresol.

20. A process according to claim 14, where the inhibitor mixture comprises <1% resorcinol, by weight.
